(19) United States
(12) Patent Application Publication
(10) Pub. No.: US 2022/0304780 A1
(43) Pub. Date: Sep. 29, 2022

(54) DEVICE AND METHOD FOR THE RETRIEVAL OF OSSEOINTEGRATED DENTAL IMPLANTS BY COMBINING THERMAL ENERGY AND ULTRASONIC VIBRATION

(71) Applicant: UNIVERSIDAD DE LOS ANDES, Las Condes (CL)

(72) Inventor: Ziyad Samir HAIDAR, Las Condes (CL)

(21) Appl. No.: 17/629,119

(22) PCT Filed: Jul. 23, 2020
(86) PCT No.: PCT/IB2020/056978

§ 371 Date: Jan. 21, 2022

(30) Foreign Application Priority Data
Jul. 23, 2019 (EP) 19187923.8

(51) Int. Cl. A61C 8/00; A61C 3/03; A61B 18/08

(52) U.S. Cl. CPC A61C 8/0089; A61C 3/03; A61B 18/082; A61B 2018/00321

(57) ABSTRACT

Device and method for the retrieval of osseointegrated dental implants by combining thermal energy and ultrasonic vibration.

### FIELD OF THE INVENTION

The present invention relates generally to devices and/or instruments for the controlled exertion of dental implants from the jawbone. More specifically, the invention relates to implant exertion devices which combine different energy sources to aid in demineralizing and loosening the implant-bone interface resulting in a rapid, minimally-invasive and non-traumatic retrieval of the integrated screw of the implant.

The present invention also refers to a method or system for the retrieval of an osseointegrated dental implant by using a combination of thermal energy and vibration energy.

### STATE OF THE ART

Dental implants have changed the field of Dentistry. Dental implants are designed to be a permanent replacement for missing tooth or teeth and are a popular alternative to removable dentures or fixed bridges. Implants anchor artificial teeth directly into the jawbone (this is known as osseointegration) which makes them a more functional and aesthetically-pleasing restoration. Today, the use of dental implants is widespread. However implant success is controversial. 90%-95% has been reported as the success rate of implants over the 10 years. It's estimated that about 5 to 10 percent of dental implants fail, either shortly after a procedure or months or years later. In any case, according to the National Institutes of Health, there are over 100 million people with missing teeth, and the demand for implant dentistry is higher than ever. Indeed, at least several million dental implants are currently in the jaws of patients around the world, with an estimate of 2 million new implants being placed annually. Nonetheless, as explained above, their potential for clinical failure is yet a significant concern for both patients and dentists. As with any other surgical procedures, there is a variety of internal and external factors that cause complications or even total failure of the treatments, and dental implants are not an exception. As cited above, at least 5% to 10% of dental implants fail every year. This translates into around 200,000 to 500,000 failed implants annually. Over the time, this percentage of dental implants that fail is expected to increase, due to several biological and technical issues. Inevitably, clinicians will dedicate more time than before to deal with wrong-positioned and failing implants.

When a dental implant has to be removed, whether due to unsuccessful insertion, fracture or peri-implantitis (chronic cervical infection causing marginal bone loss and loss of control of infection around the dental implant), the surgeon faces with a lack of efficient tools designed to facilitate the unscrewing of the dental implant without destroying it or the neighboring teeth and tissues. Hence, the only way to remove them is by performing an open surgery. However, the major problem associated with this time- and cost-consuming operation is that tools strong enough to unscrew the dental implant are also not available. Thus, the surgeon is faced with the challenge of removing the dental implant with unsuited utensils (such as tools used for exodontias), thus assuming a considerable risk of overheating and/or inducing direct trauma. Alternatively, the surgeon has to grind away the jaw bone surrounding the dental implant in order to expose part of it and achieve a better grip on the dental implant so to release it from the surrounding bony structures. The latter procedure normally entails extensive bone loss, also bearing the additional risk of damaging neighboring vital structures.

Among the known methods for dental titanium implant removal which are available in the prior art are the following: counter-ratchet technique, reverse screw technique, piezo-tips, high-speed burs, elevators, forceps and trephine burs that remove peripheral bone. All of these techniques are considered to be invasive and traumatic for the patient, mainly due to the exertion of excessive and damaging mechanical forces and thermal energies. Moreover, these methods are associated with healthy bone sacrifice, thus requiring bone grafting procedures afterwards.

Therefore, there is an unmet medical need to find better solutions and devices for the safe, rapid and non-invasive retrieval of dental implants.

Precisely, the present invention is focused on solving the above cited problems by providing a device and a methodology which can be effectively and safety used in the controlled exertion of dental implants from the jawbone and that, at the same time, substantially reduce the injury risk caused by the over-heat and/or bone degeneration effects, which can even lead to mucosal and bone necrosis.

### DESCRIPTION OF THE INVENTION

### Brief Description of the Invention

According to the present invention, a process and a device have been developed which allow a fast, effective, atraumatic, safe and one-piece removal of osseointegrated dental implants.

In a preferred embodiment, the process and the device of the invention are characterized by the combined, controlled and measured application of thermal energy with ultrasonic vibrations, in order to destroy the interface or bone-implant union with the minimum damage or injury for the peri-implant tissues.

According to the present invention, the methodology and the device of the invention will confer, among others, the following advantages:
(1) Reduction of the thermal energy generated during the removal process of the implant by introducing comfortable ultrasonic vibrations for the patient.
(2) The thermal energy is specifically applied on the implant, reducing the risk of thermal injury to adjacent tissues.
(3) The need for surgical access to the implant is eliminated thus reducing the morbidity of the procedure.
(4) The need to remove healthy peri-implant bone tissue is eliminated.
(5) The mechanical force necessary to remove the implant is reduced to the minimum removal torque thus reducing the risk of fracture of the bone and/or implant.

It is thus an object of the present invention to provide a device and/or instrument capable to control the application of combined and safe energies (thermal and vibration energy) to aid in releasing the implant-bone interface (for example by means of the dissolution, demineralizing or breaking-down the implant-bone interface) resulting in a rapid, non-traumatic and sound retrieval of the osseointegrated screw from the jaw bone, with minimal removal torque and thus with minimal or diminished necrosis/loss of surrounding bone.

Said object is preferably achieved by means of a device for the retrieval of an osseointegrated dental implant comprising, at least:
an adapter configured for its attachment to said dental implant by means of a threaded connection;
an application head connected to the adapter, which is equipped with one or more housings adapted to receive at least one first source of thermal energy and at least one second source of vibration energy;
a temperature sensor;
wherein the reception channels of the application head, the central channel of the application head and the central channel of the adapter are inter-connected for achieving a localized perpendicular combined energy wave.

The temperature sensor can be either i) connected to the application head or to the adapter (for measuring the temperature in the device, in the dental implant or the surrounding tissues thereof) or ii) being a wireless temperature sensor.

As a result, the device is capable of timely combining the minimum and safest possible temperature with a comfortable and effective ultrasonic vibration frequency for the shortest required time, in order to achieve non-traumatic and sound implant retrieval by using the minimum possible torque force.

In a preferred embodiment of the present invention, the first source for applying thermal energy comprises an electro-cautery unit.

In a preferred embodiment of the present invention, the second source for applying vibration energy is adapted to operate in the ultrasound frequency band.

In yet another preferred embodiment of the present invention, the application head and the adapter of the device are coupled through a threaded connection, being preferably internally connected to the application head.

In yet another preferred embodiment of the present invention, the head and the adapter comprise coincident central and connected channels, arranged so that the energy sources can be applied inside the dental implant. This results in a more focused and localized effect on the implant, thus optimizing the extraction processes thereof.

In yet another preferred embodiment of the present invention, the temperature sensor of the device is adapted for its insertion into the housings of the head. Therefore the internal structures of the device are optimized for housing connections to every external element thereof.

In yet another preferred embodiment of the present invention, the temperature sensor is coupled to the adapter by means of a side housing arranged in said adapter.

In yet another preferred embodiment of the present invention, the device further comprises an auxiliary application arm, adapted for providing support to the thermal and/or vibration sources when they are applied to the device. The auxiliary application arm can be used for improving stability and precision during the delivery of energy to the implant through the thermal/vibration first and second energy sources.

In yet another preferred embodiment of the present invention, the thermal and vibration energy sources are combined for their joint application through one of the housings of the device. More preferably, both sources of energy are coupled through a ceramic isolation element and applied to the head as a combined energy source.

A further object of the invention relates to a device according to any of the embodiments described in the present document, for use in the retrieval of an osseointegrated dental implant.

A further object of the invention relates to a system for the retrieval of an osseointegrated dental implant, comprising a device according to any of the embodiments described in the present document, combined with at least a first source of thermal energy and a second source of vibration energy.

A further object of the invention relates to a system according to any of the embodiments described in the present document, for use in the retrieval of an osseointegrated dental implant.

A further object of the invention relates to a method for preparing an artificial dental implant prior to its insertion in a jawbone, comprising the use of a device or a system according to any of the embodiments described in the present document, comprising the realization of at least the following steps:
attaching the adapter of the device to said dental implant by means of a threaded connection;
connecting the first source of thermal energy and at the second source of vibration energy to the application head connected to the adapter;
measuring the temperature in the device or in the dental implant with the temperature sensor connected to the application head or to the adapter. A real-time monitoring of temperature changes is carried out in the jawbone (apical part of dental implant).

In a preferred embodiment of the present invention, the method further comprises the application of thermal energy and vibration energy to the dental implant by means of the first thermal energy source and the second vibration energy source of the device.

### DESCRIPTION OF THE FIGURES

The characteristics and advantages of this invention will be more apparent from the following detailed description, when read in conjunction with the accompanying drawings, in which:

FIG. 1 represents a schematic view of the device according to the invention, depicting an adapter configured for its mechanical attachment to a dental implant, an application head equipped with a plurality of housings configured to receive one or more thermal and/or vibration energy sources, a temperature sensor, and an auxiliary application arm adapted for providing support to said thermal and/or vibration energy sources.

FIG. 2 represents a schematic view of an alternative embodiment of the invention, wherein the temperature sensor is directly coupled to the adapter of the device, by means of a side housing adapted for this purpose, and wherein reception channels (5′), the central channel of the application head (8) and the central channel of the adapter (8′) are inter-connected.

FIGS. 3a and 3b represent two perspective views of the retrieval device, according to the embodiments of the invention described of FIG. 1 and FIG. 2, respectively.

FIG. 4 represents a detail view of the application head of the retrieval device, according to a preferred embodiment of the invention.

FIGS. 5a and 5b represent detail views of the retrieval device, according to two alternative embodiments of the invention. It is noteworthy that the adapter can fit into any implant type or brand, into the inner helix, rendering the device of the invention universal.

FIG. 6 is a side view and cross section of the application head wherein it is shown that the reception channels (5′), the central channel of the application head (8) and the central channel of the adapter (8′) are inter-connected. This is also shown in FIG. 2.

FIG. 7 is a side view and cross section of the adapter wherein two alternative preferred embodiments are represented: A) conical morphology of the adapter internal channel and B) adapter internal channel with two rectangular sections. The upper section is wider as compared with the bottom section of the channel.

### NUMERICAL REFERENCES USED IN THE FIGURES

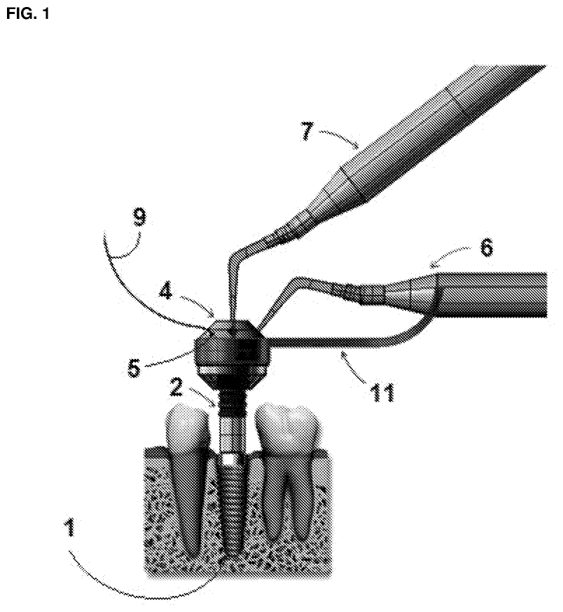

In order to provide a better understanding of the technical features of the invention, FIGS. 1-6 are accompanied of a series of numeral references which, with an illustrative and non limiting character, are hereby represented:

| | |
|---|---|
| (1) | Dental implant. |
| (2) | Adapter of the device to the dental implant. |
| (3) | Internal screw connection of the implant. |
| (4) | Application head. |
| (4′) | Threaded connection between the application |
| | head (4) and the adapter (2). |
| (5) | Housings. |
| (5′) | Reception channels. |
| (6) | First source for the application of thermal energy. |
| (7) | Second source for the application of vibration energy. |
| (8) | Central channel of the application head. |
| (8′) | Central channel of the adapter. |
| (9) | Temperature sensor. |
| (10) | Side housing of the adapter. |
| (11) | Auxiliary application arm. |
| (12) | Conical morphology of the adapter internal channel |
| | (alternative embodiment). |
| (12′) | Adapter internal channel with two rectangular sections. |
| | The upper section is wider as compared with the bottom |
| | section of the channel (alternative embodiment). |

### DETAILED DESCRIPTION OF THE INVENTION

In the following description, for purposes of explanation and not limitation, details are set forth in order to provide a thorough understanding of the present invention. However, it will be apparent to those skilled in the art that the present invention may be practiced in other embodiments that depart from these details and descriptions without departing from the spirit and scope of the invention. Certain embodiments will be described below with reference to the drawings wherein illustrative features are denoted by reference numerals.

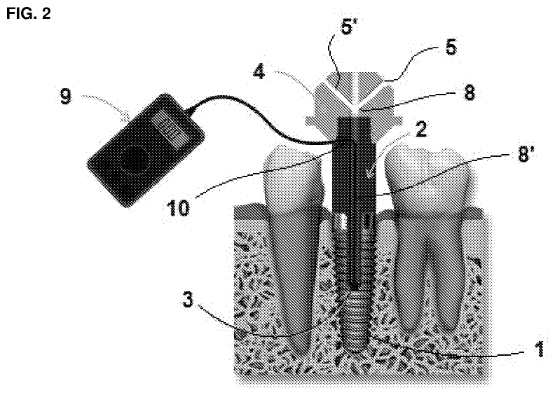

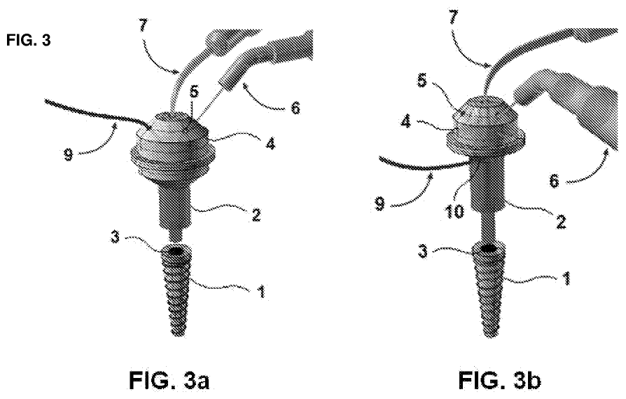

As shown in FIGS. 1-2 of the present document, the device for the retrieval of dental implants (1) of the invention preferably comprises an adapter (2) configured for its mechanical attachment to a dental implant (1), and more preferably to the portion of said dental implant (1) intended to be housed in the jawbone of a subject. The fixing or coupling of the adapter (2) to the implant (1) is preferably carried out by means of a threaded connection (3), as shown in the FIGS. 3a-3b. Thus, in an example of application of the device for the extraction of an implant (1), it is possible to first remove the external elements associated therewith, such as the abutment or dental crown (not shown in the figures), and use the internal threaded connection (3) of the implant (1) to house the adapter (2). In this way, an even stronger connection is achieved between the extraction device and the implant (1), which enables retrieval operations to be carried out with greater precision and safety.

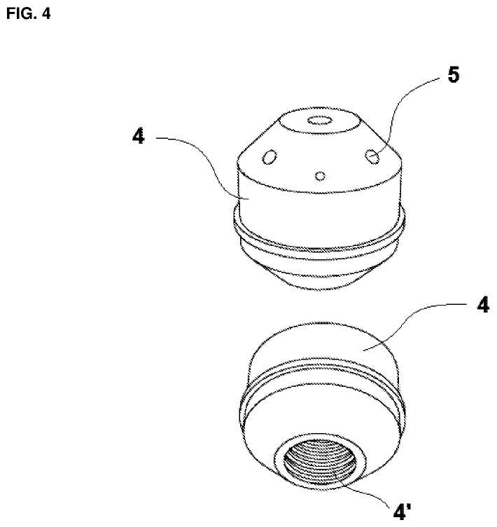

The adapter (2) is also connected to an application head (4) comprised in the device, which is equipped with a plurality of housings (5) configured to receive thermal and vibration energy (6, 7). The application head (4) and the adapter (2) can be coupled, for example, through a threaded connection (4′) (FIG. 4). Preferably, the energy application sources (6, 7) comprise at least one first source (6) for applying thermal energy (for example, an electro-cautery unit) and at least one second source (7) for applying vibration energy (for example, a source of vibration in the ultrasound frequency band). Said sources (6, 7) are shown in FIGS. 1-2, by way of example of a preferred embodiment of the invention.

The main mode of operation of the device of the invention is thus based on its fixation inside the dental implant (1), once its external elements (abutment and/or dental crown) have been removed therefrom. Once the device has been fixed to the implant (1), it is possible to subsequently apply the combination of thermal (6) and vibration (7) sources in the head (4) through the housings (5) arranged for this purpose. Said housings (5) preferably comprise connected channels (5′) (FIG. 2) for receiving the first (6) and second (7) sources, so that said sources (6, 7) can be introduced at a certain depth in the application head and/or in the adapter (2) itself towards the implant (1). The application of a combined energy source (thermal and vibration energy) to the implant (1) itself and to the surrounding tissues can thus be achieved.

In a preferred embodiment of the invention, the head (4) and the adapter (2) comprise coincident central channels (8, 8′), so that the energy sources (6, 7) can be applied inside the dental implant (1). This results in a more direct effect on the implant (1), thus optimizing the extraction processes thereof.

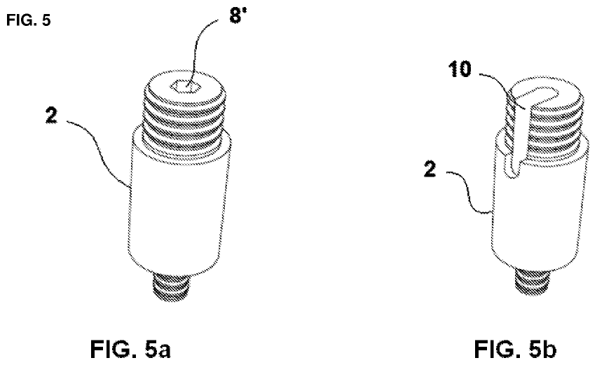

In a preferred embodiment of the invention, the device further comprises a temperature sensor (9) configured for its insertion into the housings (5) of the head (4), as shown in FIGS. 3a and 5a. Alternatively, the sensor may be directly coupled to the adapter (2) of the device by means of a side housing (10) adapted for this purpose (FIGS. 3b and 5b). The function of the temperature sensor (9) is mainly to control the risk of injury to the tissues near the dental implant (1) so that, when the temperature in the device reaches a certain threshold value, it is possible to regulate the application of sources (6, 7) of thermal and/or vibratory energy.

In a further embodiment of the invention, the device can be equipped with an auxiliary application arm (11) (FIG. 1) which is adapted for providing support to the thermal (6) and/or vibration (7) sources while they are used for the retrieval of a dental implant (1). The auxiliary application arm (11) can thus be used for improving stability and precision to the delivery of energy to the implant (1) through the thermal/vibration sources (6, 7).

In a further embodiment of the invention, the thermal (6) and vibration (7) energy sources can be combined for their joint application through one of the housings (5) of the device.

A further object of the invention refers to a system comprising a device according to any of the embodiments described herein, combined with at least a thermal energy source (6) and a vibration energy source (7).

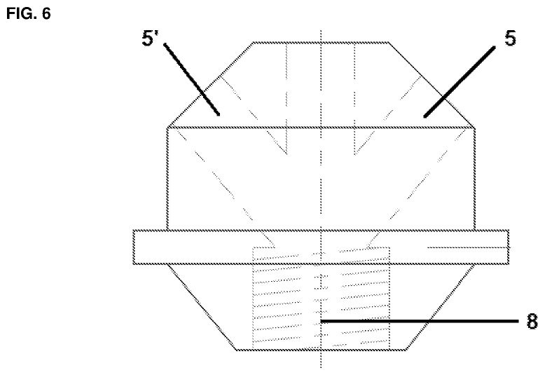

In a preferred embodiment, the reception channels of the application head (5′), the central channel of the application head (8) and the central channel of the adapter (8′) are inter-connected (see FIG. 6 and FIG. 2). This inter-connexion between 5′, 8 and 8′ has been designed for achieving a localized perpendicular combined energy wave which aids in localizing the thermal energy beam apically to the full length of the implant (localized and contained wave-shock like) while exerting mechanical vibrations (coronally-apically and side-ways) to aid in breaking the bone-implant interface lock in synchronization with demineralizing.

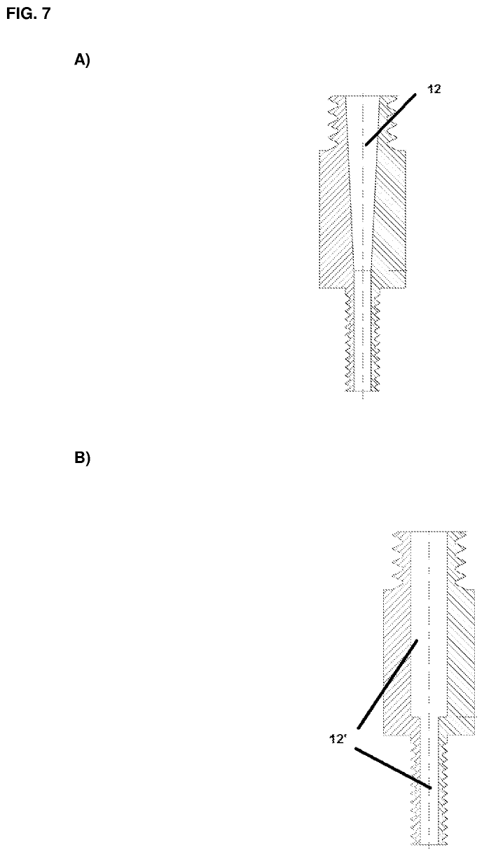

In a preferred embodiment, the adapter internal channel has a conical morphology or a rectangular morphology with two sections (the upper section is wider as compared with the bottom section) (see FIG. 7). These preferred morphologies have been designed for achieving a localized perpendicular combined energy wave which aids in localizing the thermal energy beam apically to the full length of the implant (localized and contained wave-shock like) while exerting mechanical vibrations (coronally-apically and side-ways) to aid in breaking the bone-implant interface lock in synchronization with demineralizing.

### EXAMPLES

### Example 1. Individual Application of Thermal Energy or Ultrasonic Vibrations in Dental Implants without the Device of the Invention

This example has the purpose of characterizing the magnitude/distribution of thermal energy by using an electrosurgical unit or ultrasound individually on dental implants. In order to do this, the tip of the electrosurgical unit/ultrasound was placed in the center of the implant and the corresponding stimulus was applied for 5, 10 or 15 seconds (maximum allowed by the electrosurgical unit). The magnitude or distribution of thermal energy was recorded with a thermal camera.
5 seconds:

Ultrasound: The ultrasound not only produced the desired vibrations, but also generated secondary thermal energy. The thermal energy was concentrated in the implant; generating a sudden increase of the temperature of the implant (20.53° C.-39.81° C. max) and the peri-implant bone at the cortical level (average 25.44° C. vs. basal 20.47° C.). The temperatures reached in the bone/implant interface are well below to those necessary to induce a desired effect.

Electrosurgical unit: The use of electrosurgical unit produced a slight increase in the temperature of the implant concentrated mainly at apical level (23° C.-28.5° C. max, delta T°=5.5° C.). The apical peri-implant bone reached a maximum temperature of only 26.9° C., which remains low to induce the desired effects.
10 seconds:

Ultrasound: Again the generation of vibrations and secondary thermal energy was observed. The thermal energy was concentrated in the implant, generating an abrupt increase of the temperature of the implant (20.84° C.-33.97° C. max, delta of T°=13.13° C.) and of the peri-implant bone at the cortical level (27.91° C. average vs. 21.73° C. basal). The temperatures reached in the bone/implant interface are well below those necessary to induce effects with therapeutic potential.

Electrosurgical unit: The application of electrosurgical unit produced a slight increase in the temperature of the implant concentrated mainly at the apical level (24° C.-31.63° C. max). The increase in time from 5 seconds to 10 seconds generated a greater temperature (31.6° C. max-24° C.=7.6° C.), however it remains insignificant for potential clinical applications. The greatest effects continue to be seen mainly in the apical part with poor distribution to the rest of the bone/implant interface.
15 Seconds:

Ultrasound: The ultrasound not only produced the desired vibrations, but also generated secondary thermal energy. The thermal energy was concentrated in the implant; generating a sudden increase in the temperature of the implant and of the peri-implant bone mainly at the cortical level. Although sufficient temperatures were reached to induce bone necrosis, thermal energy was concentrated mainly in the marginal cortex; poorly propagating to the rest of the bone/implant interface (apex). Considering that the basal temperature at the time of application was 20.47° C. average, the temperature that could be reached in vivo in a bone at 37° C. is a concern. On the other hand, the fact that thermal energy is concentrated mainly at the cortical level and poorly diffuses to the rest of the bone/implant union reduces the therapeutic effect of its application.

Electrosurgical unit: In contrast, the use of the electrosurgical unit produced a greater dissipation/propagation of the thermal energy through the bone/implant junction, showing greater homogeneity in the temperatures registered in the cortex vs Apex. Despite of this, the temperatures reached are low enough to generate the desired effects (mainly due to the low power of the equipment and the limitation of the manufacturer in terms of the maximum application time of only 15 seconds). Higher power/time of application of the electrosurgical unit could increase the thermal energy to clinically useful values, however, it concerns the uncontrolled dissipation of thermal energy to the peri-implant tissues (given that in the case of monopolar electrosurgical unit the thermal energy diffuses from the active pole to the receiver).

### Example 2. Individual Application of Thermal Energy or Ultrasonic Vibrations with the Device of the Invention

The previous experiment was repeated using the device of the invention, but by applying individual energy. In this case it was decided to apply individually 15 seconds of ultrasound or electrosurgical unit since the intention was to generate the maximum thermal energy. The purpose was to characterize the magnitude/distribution of thermal energy by using electrosurgical unit or ultrasound individually through the device of the invention. The device of the invention allowed the free transmission of the ultrasonic vibrations from the ultrasound to the implant. The invention device controls or guides or focuses the propagation of the vibrational as well as heat beam cortical to apex. The device allowed the free transmission and dissipation/homogeneous propagation of the thermal energy of the electrosurgical unit (main source of thermal energy generation in this model) to the whole bone/implant interface (cortical vs. apex).

### Example 3. Combined Application of Thermal Energy or Ultrasonic Vibrations with the Device of the Invention

When the temperatures reached in the bone/implant interface are below to those necessary to induce a therapeutic effect, in a preferred embodiment both thermal and vibration energy are combined to reach the desired temperature.

The objective is to maintain at least 45° C. within the implant itself and throughout its length within the bone, for the longest time possible, as this accumulative temperature is safe and comfortable for the patient, and effective in demineralizing the bone-implant interface. Specifically the objective is to maintain said 45° C. until the titanium fixture/screw/implant is retrieved from the bone.

The specific protocol for reaching this temperature by combining thermal and ultrasound energies directly depend on different factors, for example the equipment used (brand, quality, efficiency, power, resiliency) in the clinical setting.

Nevertheless, just with the objective of proving one of the possible protocols for reaching the desired temperature, this example shows the serial and combined use of the electrosurgical unit and ultrasound in cycles of 10 to 20 seconds (preferably 15-second cycles) interrupted by 25 to 35 seconds of rest (preferably 30 seconds of rest). Given the limitations of the electrosurgical unit, it was decided to increase the thermal energy generated by a serial or stepped application strategy. To do this, the electrosurgical unit or ultrasound was applied in cycles (10 cycles, each of 15 seconds of application followed by 30 seconds of rest). The purpose was to characterize the magnitude/distribution of thermal energy when applying the device following this new application strategy.

The combined application of thermal energy and ultrasonic vibrations following a stepped or serial strategy with the device of the invention allowed the generation of enough thermal energy to induce temperatures with potential biological/therapeutic effect on the implant and peri-implant alveolar bone. The goal of maintaining at least 45° C. within the implant itself and throughout its length within the bone, for the longest time possible, was achieved.

According to the records of the internal thermocouple of the device of the invention, the temperature at implant level rose by 17.1° C. average. It is considered that in in vivo conditions the basal temperature of the bone will be of 37° C.; said increase of temperature will allow reaching temperatures above 45° C. but below 51.6° C., always in agreement with good clinical practices (GCP) in order to avoid bone tissue damage/necrosis. Regarding the distribution of this thermal energy, it is confirmed that the device of the invention allows concentrating and localizing the thermal energy in a homogeneous way in the bone/implanting interface, reducing the amount of thermal energy which is dissipated to neighboring tissues, and therefore increasing the safety of the protocol by decreasing the area of bone tissue exposed to thermal injury.

In conclusion, the use of the device of the invention to apply, in combination, ultrasounds and the electrosurgical unit gives rise to the following results:
The device of the invention transmits the ultrasound ultrasonic vibrations directly to the implant and peri-implant bone.
The device of the invention minimizes the amount of thermal energy secondary to vibration (unwanted effect which is dangerous because it induces a sudden and non-homogeneous temperature increase in the implant and cortical bone).
The device of the invention freely transmits the thermal energy of the electrosurgical unit to the implant and peri-implant tissue. The electrosurgical unit is therefore the main source of thermal energy when applying this strategy.
The application of thermal energy and ultrasonic vibrations with the device of the invention not only allows to generate enough thermal energy to induce temperatures with potential biological/therapeutic effect, but also concentrates and locates said thermal energy in the implant and peri-implant bone (1 mm, preferably 0.2-0.3 mm). This reduces the risk of thermal injury due to the uncontrolled dissipation of thermal energy on the tissues adjacent to the implant, thus increasing the overall safety of the protocol.
Last but not least, as the combined energy is applied perpendicularly, it aids in localizing the thermal energy beam apically to the full length of the implant (localized and contained wave-shock like) while exerting mechanical vibrations (coronally-apically and side-ways-micro/macro vibrations or cracks) to aid in breaking the bone-implant interface lock in synchronization with demineralizing.

1. System for the retrieval of an osseointegrated dental implant (1) comprising a device which in turn comprises:
an adapter (2) configured for its attachment to said dental implant (1) by means of a threaded connection (3);
an application head (4) connected to the adapter (2), which is equipped with one or more housings (5) adapted to receive at least one first source (6) of thermal energy in combination with at least one second source (7) of vibration energy;
a temperature sensor (9);
wherein the reception channels (5′) of the application head (4), the central channel (8) of the application head (4) and the central channel (8′) of the adapter (2) are inter-connected to localize the thermal energy beam apically to the full length of the implant (1) while exerting mechanical vibrations to aid in breaking the bone-implant interface lock;
characterized in that the device is combined with at least a first source (6) of thermal energy and second source (7) of vibration energy.

2. System, according to claim 1, wherein the adapter central channel (8′) has i) a conical morphology (12), or ii) a rectangular morphology (12′) with two sections, the upper section being wider as compared with the bottom section.

3. System according to the preceding claims, wherein the first source (6) for applying thermal energy comprises an electro-cautery unit and wherein the second source (7) for applying vibration energy is adapted to operate in the ultrasound frequency band.

4. System according to any of the preceding claims, wherein the application head (4) and the adapter are coupled through a threaded connection (4′).

5. System according to any of the preceding claims, wherein the head (4) and the adapter (2) comprise coincident central channels (8, 8′), arranged so that the energy sources (6, 7) can be applied longitudinally or vertically inside and throughout the length of the dental implant (1).

6. System according to any of the preceding claims, wherein the thermal (6) and vibration (7) energy sources are combined for their joint application through one of the housings (5) of the device.

7. System according to the preceding claim, wherein the first (6) and second (7) energy sources are coupled through a ceramic isolation element.

8. System according to any of the preceding claims, for use in the retrieval of an osseointegrated dental implant (1).

9. Method for preparing a system according to any of the claims 1 to 7 which comprises:
attaching the adapter (2) of the device to the dental implant (1) by means of a threaded connection (3);
connecting the first source (6) of thermal energy and at the second source (7) of vibration energy to the application head (4) connected to the adapter (2);
connecting the temperature sensor (9).
